SEQUENCE LISTING (48 sequences)

SEQ ID NO: 1 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 882 aa, Teseptimavirus T7
NTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - protein, 889 aa, synthetic construct
MHHHHHHNTINIAKNDFSDIELAAIPFNTLADHYGERLAREQLALEHESYEMGEARFRKMFERHLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 4 - protein, 889 aa, synthetic construct
MHHHHHHNTINIAKNDFSDIELAAIPFNTLADHYGERLAREQLALEHESYEMGEARFRKMFERQLKAGPVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 5 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMCWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 6 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSFFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 7 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKFFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 8 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIKESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 9 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYMEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 10 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHAQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 11 - protein, 889 aa, synthetic construct
MHHHHHHNTINIAKNDFSDAELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 12 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAPYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 13 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLETTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 14 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGMAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 15 - protein, 889 aa, synthetic construct
MHHHHHHNTINIAKADFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGMAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 16 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQNVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 17 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQKRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 18 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRFGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 19 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALCGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 20 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYGMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 21 - protein, 889 aa, synthetic construct
MHHHHHHNTINIAKNDFSDNELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 22 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIQESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 23 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSDFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 24 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALLAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 25 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRVGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 26 - protein, 889 aa, synthetic construct
MHHHHHHNTINIAKADFSDNELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQNVLEETIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHAQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 27 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIQESVSVTVVAAVEAMNWLKSAAKLLAAEVKDKKTGEILRKRCAVHWVTPDGFPVWQEYKKPIQTRLNLMFLGQKRLQPTINTNKDSEIDAHAQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 28 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRFGALCGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYGMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHAQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 29 - protein, 889 aa, synthetic construct
MHHHHHHNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRVGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALLAWKRAAAAVYRKDKARKSRRISLEFMLEQANKFANHKAIWFPYGMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHAQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 30 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 31 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcatcttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 32 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtccagttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 33 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatgtgttggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 34 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcatttttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 35 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaatttttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 36 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgattaaagaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 37 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacatggaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 38 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacgcacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 39 - DNA, 2670 bp, synthetic construct
  source 1..2670: mol_type = other DNA
atgcaccatcaccatcaccataacacgattaacatcgctaagaacgacttctctgacgcagaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 40 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatggctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 41 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaaacaaccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 42 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctccatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 43 - DNA, 2670 bp, synthetic construct
  source 1..2670: mol_type = other DNA
atgcaccatcaccatcaccataacacgattaacatcgctaaggcagacttttctgacaacgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagaattcggcttccgtcaaaatgtcctggaagaaaccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacgcacaggagtctggtatcgctcctaactttgtacacagccaagacggtagccaccttcgtaagactgtggtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 44 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcacagcgtggaaacgtgctgccgctgctgtgtaccgcaaggacaaggctcgcaagtctcgccgtatcagccttgagttcatgcttgagcaagccaataagtttgctaaccataaggccatctggttcccttacaatatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgattcaggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagaaacgcttacagcctaccattaacaccaacaaagatagcgagattgatgcacacgcacaggagtctggtatcgctcctaactttgtacacagccaagacggtagccaccttcgtaagactgtggtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 45 - DNA, 2670 bp, synthetic construct
  source 1..2670: mol_type = other DNA
atgcaccatcaccatcaccataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtttcggtgcgctgtgtggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcacagcgtggaaacgtgctgccgctgctgtgtaccgcaaggacaaggctcgcaagtctcgccgtatcagccttgagttcatgcttgagcaagccaataagtttgctaaccataaggccatctggttcccttacggaatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacgcacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 46 - DNA, 2670 bp, synthetic construct
  source 1..2670: mol_type = other DNA
atgcaccatcaccatcaccataacacgattaacatcgctaagaacgacttttctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaaacgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgtaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcctggcgtggaaacgtgctgccgctgctgtgtaccgcaaggacaaggctcgcaagtctcgccgtatcagccttgagttcatgcttgagcaagccaataagtttgctaaccataaggccatctggttcccttacggaatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacgcacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 47 - protein, 21 aa, synthetic construct
  VARIANT 2..21: note = This region may encompass 0-20 residues
MHHHHHHHHHHHHHHHHHHHH

SEQ ID NO: 48 - protein, 6 aa, synthetic construct
HHHHHH

(19) United States
(12) Patent Application Publication
(10) Pub. No.: US 2025/0019675 A1
(43) Pub. Date: Jan. 16, 2025

(54) T7 RNA POLYMERASE VARIANTS FOR RNA SYNTHESIS

(71) Applicant: GLAXOSMITHKLINE BIOLOGICALS SA, Rixensart (BE)

(72) Inventors: Brent M. DORR, Collegeville, PA (US); Joel MELBY, Boston, MA (US); Deepak RUDRAPPA, Collegeville, PA (US); Jeliazko JELIAZKOV, Collegeville, PA (US); Nilesh VAIDYA, Rockville, MD (US)

(73) Assignee: GLAXOSMITHKLINE BIOLOGICALS SA, Rixensart (BE)

(21) Appl. No.: 18/684,923

(22) PCT Filed: Aug. 30, 2022
(86) PCT No.: PCT/IB2022/058118

§ 371 Date: Feb. 20, 2024

Related U.S. Application Data
(60) Provisional application No. 63/239,458, filed on Sep. 1, 2021.
(60) Provisional application No. 63/395,070, filed on Aug. 4, 2022.

(51) Int. Cl. C12N 9/12; C12P 19/34

(52) U.S. Cl. CPC C12N 9/1247; C12P 19/34; C12Y 207/07006

(57) ABSTRACT

The present disclosure provides T7 RNA polymerase variants, the use of which increases transcription efficiency and improves the quality and yield of the enzyme. Also provided are polynucleotides encoding the T7 RNA polymerase variants, host cells capable of expressing the T7 RNA polymerase variants, and methods of using the T7 RNA polymerase variants for high-efficiency transcription.

### CROSS-REFERENCE TO RELATED APPLICATIONS

This application is filed pursuant to 35 U.S.C. § 371 as a United States National Phase Application of International Application No. PCT/IB2022/058118 filed on Aug. 30, 2022, which claims the benefit of U.S. provisional application No. 63/239,458 filed on Sep. 1, 2021, and U.S. provisional application No. 63/395,070 filed on Aug. 4, 2022.

### SEQUENCE LISTING

The instant application contains a Sequence Listing which has been submitted electronically in XML format and is hereby incorporated by reference in its entirety. Said XML copy, created on Jan. 18, 2024, is named 70034US03_SL.xml and is 113,680 bytes in size.

### FIELD OF THE INVENTION

The present disclosure relates to improved T7 RNA polymerase variants and their use in RNA synthesis. The invention also includes polynucleotides encoding the improved T7 RNA polymerase variants and host cells comprising said polynucleotides. The invention also includes methods of using the T7 RNA polymerase variants for high-efficiency transcription.

### BACKGROUND OF THE INVENTION

RNA polymerases are a class of enzymes responsible for transcribing a DNA template into RNA transcripts during the process of transcription. Certain bacteriophage-derived RNA polymerases, including T7 polymerase, T3 polymerase, and SP6 polymerase, exhibit high specificity for their cognate promoters. For example, the T7 bacteriophage-derived RNA polymerase (T7 RNA polymerase, or “T7 RNAP”) transcribes DNA only downstream of a T7 promoter sequence. (Sousa et al., Model for the mechanism of bacteriophage T7 RNAP transcription initiation and termination. 1992; J Mol Biol. 224(2): 319-34).

Isolated DNA-dependent RNA polymerases are useful for producing RNA by the process of in vitro transcription (IVT). In a typical IVT reaction, a DNA template is engineered to include a bacteriophage promoter sequence (e.g., from the T7 coliphage) upstream of the sequence of interest, followed by transcription using the corresponding RNA polymerase.

In addition to producing full-length, single-stranded RNA copies of the DNA template, IVT reactions can also produce contaminating RNA species, such as truncated transcripts (shorter than full length), run-on transcripts (longer than full length), and double-stranded RNA (dsRNA). Such contaminants can negatively impact downstream applications of the RNA and can necessitate additional purification steps.

Therefore, there is a need for improved T7 RNA polymerase variants that when used in an in vitro transcription reaction, increase transcription efficiency while reducing the number of double-stranded RNA contaminants, truncated transcripts, and run-on transcripts produced during the in vitro transcription reaction.

### SUMMARY OF THE INVENTION

The present disclosure provides novel T7 RNA polymerase variants and in vitro transcription methods using these variants. The T7 RNA polymerase variants of the present disclosure, when used in an in vitro transcription reaction, significantly improve the quality and yield of the RNA transcript. Some of these improvements include one or more of the following: increasing transcription efficiency, increasing the yield of RNA, improving the fidelity of transcription, and reducing the amount of dsRNA contamination, among other things.

In one aspect of the present disclosure, T7 RNA polymerase variants comprising at least one amino acid substitution and/or modification relative to wild-type T7 RNA polymerase are provided. Thus, the present invention provides engineered T7 RNA polymerases, i.e. the T7 RNA polymerases that have been modified in a manner that would not otherwise exist in nature.

In another aspect of the present disclosure, T7 RNA polymerase variants comprising at least two, at least three, at least four, at least five, at least six, at least 10, or at least 20 substitutions or modifications relative to wild-type T7 RNA polymerase are provided. In another aspect of the present disclosure provided are T7 RNA polymerase variants comprising at least two, at least three, at least four, at least five, at least six, at least 10, or at least 20 substitutions or modifications relative to wild-type T7 RNA polymerase, wherein the wild-type T7 RNA polymerase comprises an N-terminal M-Polyhistidine tag.

In a further aspect of the present disclosure, polynucleotides encoding the T7 RNA polymerase variants of the present disclosure are provided. Also provided are compositions, vectors, and host cells comprising said polynucleotides.

In a further aspect of the present disclosure, kits comprising the T7 RNA polymerase variants of the present disclosure are provided.

In a further aspect of the present disclosure, methods of performing in vitro transcription (IVT) reactions using the T7 RNA polymerase variants of the present disclosure are provided.

### BRIEF DESCRIPTION OF THE FIGURES

FIG. 1: FIG. 1 depicts capillary gel electrophoresis analysis of RNA transcripts produced by the wildtype T7 RNA polymerase in IVT reactions run as described in Example 3. The LM peak shows an internal standard included in each reaction according to the manufacturer's specifications, while the peak at 9000 nt shows the full-length RNA generated during the IVT reaction. Smaller peaks between 100 and 9000nt show premature truncation products, and degradation products formed during the IVT reaction, while peaks above 9000nt correspond to residual DNA template and additional transcription side-products.

FIG. 2: FIG. 2 depicts double-strand RNA (“dsRNA”) content in RNA transcribed using the T7 RNA polymerase variants of the present disclosure. dsRNA content is determined by the J2 antibody slot blot assay. Variant 1 contains the M1L and Q64H mutations, variant 2 contains the E69P mutation, variant 3 contains the D427C mutation, variant 4 contains the M437F mutation, variant 5 contains the E649F mutation, variant 6 contains the W688K mutation, variant 7 contains the A257M mutation, variant 8 contains the K779A mutation, variant 9 contains the I20A mutation, variant 10 contains the I784M mutation, variant 11 contains the D659T mutation, and variant 12 contains the G681P mutation. WT is the wild-type T7 RNA polymerase.

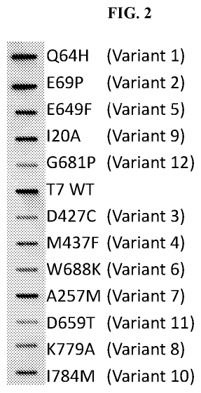

### DETAILED DESCRIPTION OF THE INVENTION

The present disclosure provides highly efficient T7 RNA polymerase variants that when used in an in vitro transcription reaction, significantly improves the quality and yield of the RNA transcript. Such efficient T7 RNA polymerase variants are achieved by engineering the T7 RNA polymerase variants.

The present disclosure provides T7 RNA polymerase variants. A T7 RNA polymerase variant is an enzyme having T7 RNA polymerase activity and at least one substitution and/or modification relative to the wild-type T7 RNA polymerase.

The present disclosure further provides T7 RNA polymerase variants that comprise multiple (two or more) amino acid substitutions and/or modifications, relative to wild-type T7 RNA polymerase. The present disclosure also provides T7 RNA polymerase variants that comprise multiple (two or more) amino acid substitutions and/or modifications relative to wild-type T7 RNA polymerase, wherein the wild-type T7 RNA polymerase comprises an N-terminal M-Polyhistidine tag.

Assays to confirm the yield and quality of RNA synthesized by the T7 RNA polymerase variants include the assays described in the examples disclosed in the present application as well as assays known in the art.

### I. Definitions

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. The term “at least one” refers to one or more.

Additionally, numerical limitations given with respect to concentrations or levels of a substance, such as solution component concentrations or ratios thereof, and reaction conditions such as temperatures, pressures, and cycle times are intended to be approximate. Unless specified otherwise, where a numerical range is provided, it is inclusive, i.e., the endpoints are included.

For the purposes of the descriptions herein, the abbreviations used for the genetically encoded amino acids are conventional and are as follows in Table 1:

TABLE 1

| AMINO ACID | THREE-LETTER | ONE-LETTER |
|---|---|---|
| ALANINE | ALA | A |
| ARGININE | ARG | R |
| ASPARAGINE | ASN | N |
| ASPARTATE | ASP | D |
| CYSTEINE | CYS | C |
| GLUTAMATE | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

When either one-letter or three-letter abbreviations are used, unless specifically preceded by an “L” or a “D” or clear from the context in which the abbreviation is used, the amino acid may be in either the L- or D-configuration about alpha-carbon (C-alpha). For example, whereas “Ala” designates alanine without specifying the configuration about the alpha-carbon, “D-Ala” and “L-Ala” designate D-alanine and L-alanine, respectively. When peptide sequences are presented as a string of one-letter or three-letter abbreviations, the sequences are presented in the N→C direction in accordance with convention.

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Amino acid” or “residue” as used in the context of the polypeptides disclosed herein refers to the specific monomer at a sequence position (e.g., P5 indicates that the “amino acid” or “residue” at position 5 is a proline.)

“Amino acid difference” or “residue difference” or “Amino acid substitution” refers to a change in the residue at a specified position of a polypeptide sequence when compared to a reference sequence.

“Aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L-His (H) it is sometimes classified as a basic residue, or as an aromatic residue as its side chain includes a heteroaromatic ring, herein histidine is classified as a hydrophilic residue or as a “constrained residue” (see below).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ions. Genetically encoded basic amino acids include L-Arg (R) and L-Lys (K).

“Conservative” amino acid substitutions or mutations refer to the interchangeability of residues having similar side chains, and thus typically involves the substitution of the amino acid in the polypeptide with amino acids within the same or similar defined class of amino acids. However, as used herein, in some embodiments, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 2 below shows exemplary conservative substitutions.

TABLE 2

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P | none |
| N, Q, S, T, Y | Other polar |
| H, Y, W, F | Other aromatic (H, Y, W, F) |
| C | None |

“Constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry, because it also has a five-membered ring.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. Methods of comparing a sequence with a specified reference sequence are known to a skilled person. For example, the Needleman Wunsch method can be used to compare any amino acid or polynucleotide sequence with a reference sequence.

“Corresponding amino acid position” is a term that is widely used and well-understood by a skilled person. A corresponding amino acid position can be identified by aligning the amino acid sequences using any of the well-known amino acid alignment methods. For example, the NCBI BLAST algorithm method can be used to identify a corresponding amino acid position. “Cysteine” or L-Cys (C) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges. The ability of L-Cys (C) (and other amino acids with −SH containing side chains) to exist in a peptide in either the reduced free −SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure L-Cys (C) is categorized into its own unique group.
“Deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, up to 20% of the total number of amino acids, or up to 30% of the total number of amino acids making up the polypeptide while retaining enzymatic activity and/or retaining the improved properties of the T7 RNA polymerase enzyme. Deletions can be directed to the internal portions and/or terminal portions of the polypeptide. In various embodiments, the deletion can comprise a continuous segment or can be discontinuous.
“Derived from” as used herein in the context of enzymes identifies the originating enzyme, and/or the gene encoding such enzyme, upon which the engineering was based. For example, the T7 RNA polymerase of SEQ ID NO: 1 was obtained by modifying the T7 RNA polymerases of SEQ ID NO: 2. Thus, the T7 RNA polymerase of SEQ ID NO: 1 is “derived from” the T7 RNA polymerase of SEQ ID NO: 2.
“Fragment” as used herein, refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99%, or more, of the full-length T7 RNA polymerase. A “functional fragment” or a “biologically active fragment”, used interchangeably, herein refers to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide. For example, a functional fragment of the T7 RNA polymerase of SEQ ID NO: 1 or SEQ ID NO: 2 is a polypeptide that has at least 50%, 60%, 70%, 80%, 90%, 95%, 99% of T7 RNA polymerase activity of SEQ ID NO:1 or SEQ ID NO: 2.
“Improved enzyme property” refers to any enzyme property made better or more desirable for a particular purpose as compared to that property found in a reference enzyme. For the T7 RNA polymerase variants described herein, the comparison is generally made to a reference T7 RNA polymerase enzyme which does not contain the particular mutation which improves enzyme efficiency. However, in some embodiments, the reference T7 RNA polymerase can be another improved T7 RNA polymerase variant.
“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, et al., 1990, J. Mol. Biol. 215: 403-410 and Altschul, et al., 1977, Nucleic Acids Res. 3389-3402). Software for performing BLAST analyses is publicly available through the National Center for Biotechnology Information website.

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel, et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided. The ClustalW program is also suitable for determining identity.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Nucleic acid” herein means a polymeric form of nucleotides of any length, which contain deoxyribonucleotides, ribonucleotides, and/or their analogs. It includes DNA, RNA and DNA/RNA hybrids. It also includes DNA or RNA analogs, such as those containing modified backbones (e.g. peptide nucleic acids (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap. RNA may be a small, medium, or large RNA. The number of nucleotides per strand of a small RNA is from 10-30 (e.g. siRNAs). A medium RNA contains between 30-2000 nucleotides per strand (e.g. non-self-replicating mRNAs). A large RNA contains at least 2,000 nucleotides per strand e.g. at least 2,500, at least 3,000, at least 4,000, at least 5,000, at least 6,000, at least 7,000, at least 8,000, at least 9,000, or at least 10,000 nucleotides per strand. The molecular mass of a single-stranded RNA molecule in g/mol (or Dalton) can be approximated using the formula: molecular mass=(number of RNA nucleotides)×340 g/mol. RNA can include, in addition to any 5′ cap structure, one or more nucleotides having a modified nucleobase. For instance, an RNA can include one or more modified pyrimidine nucleobases, such as pseudouridine and/or 5 methylcytosine residues. In some embodiments, however, the RNA includes no modified nucleobases, and may include no modified nucleotides i.e. all of the nucleotides in the RNA are standard A, C, G and U ribonucleotides (except for any 5′ cap structure, which may include a 7′ methylguanosine). In other embodiments, the RNA may include a 5′ cap comprising a 7′ methylguanosine, and the first 1, 2 or 3 5′ ribonucleotides may be methylated at the 2′ position of the ribose.

Nucleic acids can be in recombinant form, i.e., a form that does not occur in nature. For example, the nucleic acid may comprise one or more heterologous nucleic acid sequences (e.g., a sequence encoding another antigen and/or a control sequence such as a promoter or an internal ribosome entry site). The nucleic acid may be part of a vector i.e., part of a nucleic acid designed for transduction/transfection of one or more cell types. Vectors may be, for example, “expression vectors,” which are designed for expression of a nucleotide sequence in a host cell, or “viral vectors,” which are designed to result in the production of a recombinant virus or virus-like particle.

“Nucleoside triphosphate” as used herein with reference to RNA relates to standard A, C, G and U nucleosides and/or modified nucleosides including modified nucleobases, unless expressly specified otherwise.

“RNA” (or “ribonucleic acid”) as used herein relates to a molecule which comprises ribonucleotide residues. The term “ribonucleotide” refers to a nucleotide containing ribose as its pentose component. The term “RNA” comprises double-stranded RNA, single stranded RNA, isolated RNA, synthetic RNA, recombinantly generated RNA, ribo-oligonucleotides (shorter RNA sequences generally in the range of 3 to 40 nucleotides), self-amplifying RNA (“saRNA”) also referred to as self-replicating RNA or self-amplifying/replicating mRNA (“SAM”), and modified RNA which differs from naturally occurring RNA by addition, deletion, substitution and/or alteration of one or more nucleotides. Nucleotides in RNA molecules can comprise non-standard nucleotides, such as non-naturally occurring nucleotides or chemically synthesized nucleotides or deoxynucleotides. “mRNA” (or messenger RNA”) as used herein means “messenger-RNA” and relates to a transcript which is generated by using a DNA template and encodes a peptide or protein. Typically, mRNA comprises a protein coding region flanked by a 5′-UTR and a 3′-UTR. The term “antisense-RNA” relates to single-stranded RNA comprising ribonucleotide residues, which are complementary to the mRNA. The term “siRNA” means “small interfering RNA”, which is a class of double-stranded RNA-molecules comprising about 20 to about 25 base pairs.

“T7 RNA polymerase” is an RNA polymerase from the T7 bacteriophage that catalyzes the formation of RNA from DNA in the 5′→3′ direction. T7 RNA polymerase is highly specific for the T7 promoter. The T7 RNA polymerases of the present invention have T7 RNA polymerase activity, i.e. the polymerases transcribe DNA specifically starting at a T7 promoter.

“In vitro transcription (IVT)” is a procedure that allows for template-directed synthesis of RNA molecules of any sequence from short oligonucleotides to those of several kilobases in g to mg quantities. It is based on the engineering of a template that includes a bacteriophage promoter sequence (e.g. from the T7 coliphage) upstream of the sequence of interest followed by transcription using the corresponding RNA polymerase.

“Transcription efficiency” herein refers to RNA yield, and/or RNA quality, and/or rate of transcription. “Highly efficient T7 RNA polymerase variants/enzymes” are the T7 RNA polymerase enzymes/and or variants that when used in an in vitro transcription reaction, significantly improve the transcription efficiency.

“Purification” or “purifying” herein means the process of removing components from a composition or host cell or culture, the presence of which is not desired. Purification is a relative term and does not require that all traces of the undesirable component be removed from the composition. In the context of vaccine production, purification includes such processes as centrifugation, dialyzation, ion-exchange chromatography, and size-exclusion chromatography, affinity-purification, or precipitation. Thus, the term “purified” does not require absolute purity; rather, it is intended as a relative term. A preparation of substantially pure nucleic acid or protein can be purified such that the desired nucleic acid, or protein, represents at least 50% of the total nucleic acid content of the preparation. In certain embodiments, a substantially pure nucleic acid, or protein, will represent at least 60%, at least 70%, at least 80%, at least 85%, at least 90%, or at least 95% or more of the total nucleic acid or protein content of the preparation. Immunogenic molecules or antigens or antibodies which have not been subjected to any purification steps (i.e., the molecule as it is found in nature) are not suitable for pharmaceutical (e.g., vaccine) use.

“% RNA purity” as used herein is interchange with “% RNA integrity” and refers to the percent of RNA molecules that are full-length RNA consisting of entire sequence encoded in DNA template to the total RNA, e.g., have both the 5′ and 3′ ends. % Purity can be determined using different techniques known to a skilled person, e.g., Capillary Gel Electrophoresis, Gel Electrophoresis).

“Comprise” (“comprising” or “comprises”) as used herein is open-ended and means “including, but not limited to.” “Having” is used herein as a synonym of comprising. It is understood that wherever embodiments are described herein with the language “comprising,” such embodiments encompass those described in terms of “consisting of” and/or “consisting essentially of.”

“About” or “approximately” mean roughly, around, or in the regions of. The terms “about” or “approximately” further mean within an acceptable contextual error range for the particular value as determined by one of ordinary skill in the art, which will depend in part on how the value is measured, i.e. the limitations of the measurement system or the degree of precision required for a particular purpose. When the terms “about” or “approximately” are used in conjunction with a numerical range, it modifies that range by extending the boundaries above and below the numerical values set forth.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

In one embodiment there is provided a T7 RNA polymerase comprising an amino acid sequence that is at least 80%, 85%, 90%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid M1, 120, Q64, E69, A257, D427, M437, E649, D659, G681, W688, K779, and I784 is substituted to a different amino acid to that found at the corresponding amino acid position in SEQ ID NO:1. In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, A264, A268, T381, N425, Q655 and F761 is substituted to a different amino acid to that found at the corresponding amino acid position in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, A264, A268, T381, N425, M437, Q655, D659, G681, W688, F761 and K779 is substituted to a different amino acid to that found at the corresponding amino acid position in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein N15 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein I20 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein Q64 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein E69 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein A257 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein A264 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein A268 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein T381 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein N425 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein D427 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein M437 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein E649 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein Q655 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein D659 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein G681 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein W688 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein F761 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein K779 in SEQ ID NO:1 is substituted to a different amino acid.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein I784 in SEQ ID NO:1 is substituted to a different amino acid.

In another embodiment, provided herein is a T7 RNA polymerase comprising an amino acid sequence that is at least 80%, 85%, 90%, 95%, 96%, 97%, 98%, or 99% identical to the amino acid sequence set out in SEQ ID NO:1 or a functional fragment thereof, wherein the amino acid sequence comprises at least one feature selected from the group consisting of: I20 is substituted with A or L or V or G or M; Q64 is substituted with N or S or T or H; E69 is substituted with D or P; A257 is substituted with M or L or V or I or G; D427 is substituted with E or C; M437 is substituted with A or L or V or I or G or F; E649 is substituted with D or F; D659 is substituted with E or T; G681 is substituted with A or L or V or I or M or P; W688 is substituted with H or Y or F or K; K779 is substituted with R or A; and I784 is substituted with A or L or V or M or G.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: N15 is substituted with A or T or Y or Q or S, I20 is substituted with A or L or V or G or M or N; Q64 is substituted with N or S or T or H; E69 is substituted with D or P; A257 is substituted with M or L or V or I or G; A264 is substituted with F or V or M or L or I or G; A268 is substituted with C or M or L or I or G; T381 is substituted with L or Y or N or Q or S; N425 is substituted with G or T or Y or Q or S; D427 is substituted with E or C; M437 is substituted with A or L or V or I or G or F or D; E649 is substituted with D or F; Q655 is substituted with N or T or Y or S, D659 is substituted with E or T; G681 is substituted with A or L or V or I or M or P; W688 is substituted with H or Y or F or K or Q; F761 is substituted with K or W or Y or H; K779 is substituted with R or A; and I784 is substituted with A or L or V or M or G.

In another embodiment, provided herein is a T7 RNA polymerase comprising the amino acid sequence of SEQ ID NO:1 or a functional fragment thereof, wherein the amino acid sequence comprises at least one feature selected from the group consisting of: I20 is substituted with A; Q64 is substituted with H; E69 is substituted with P; A257 is substituted with M; D427 is substituted with C; M437 is substituted with F; E649 is substituted with F; D659 is substituted with T; G681 is substituted with P; W688 is substituted with K; K779 is substituted with A; and I784 is substituted with M.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: N15 is substituted with A; I20 is substituted with N; I20 is substituted with A; Q64 is substituted with H; E69 is substituted with P; A257 is substituted with M; A264 is substituted with F; A264 is substituted with V; A268 is substituted with C; T381 is substituted with L; N425 is substituted with G; D427 is substituted with C; M437 is substituted with F; M437 is substituted with D; E649 is substituted with F; Q655 is substituted with N; D659 is substituted with T; D659 is substituted with E; G681 is substituted with P; W688 is substituted with K; W688 is substituted with Q; F761 is substituted with K; K779 is substituted with A; and I784 is substituted with M.

In another embodiment, provided herein is a T7 RNA polymerase comprising the amino acid sequence of SEQ ID NO:1 or a functional fragment thereof, wherein the amino acid sequence comprises at least one feature selected from the group consisting of: N15 is substituted with A; I20 is substituted with N; I20 is substituted with A; Q64 is substituted with H; E69 is substituted with P; A257 is substituted with M; A264 is substituted with F; A264 is substituted with V; A268 is substituted with C; T381 is substituted with L; N425 is substituted with G; D427 is substituted with C; M437 is substituted with F; M437 is substituted with D; E649 is substituted with F; Q655 is substituted with N; D659 is substituted with T; D659 is substituted with E; G681 is substituted with P; W688 is substituted with K; W688 is substituted with Q; F761 is substituted with K; K779 is substituted with A; and I784 is substituted with M.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: Q64 is substituted with H; E69 is substituted with P; N15 is substituted with A; I20 is substituted with N; A264 is substituted with F; A264 is substituted with V; A268 is substituted with C; T381 is substituted with L; M437 is substituted with F; M437 is substituted with D; N425 is substituted with G; I20 is substituted with A; E649 is substituted with F; Q655 is substituted with N; D659 is substituted with E; G681 is substituted with P; W688 is substituted with Q; F761 is substituted with K; and K779 is substituted with A.

In another embodiment, provided herein is a T7 RNA polymerase comprising the amino acid sequence set out in SEQ ID NO:1 or a functional fragment thereof, wherein the amino acid sequence comprises at least one feature selected from the group consisting of: Q64 is substituted with H; E69 is substituted with P; N15 is substituted with A; I20 is substituted with N; A264 is substituted with F; A264 is substituted with V; A268 is substituted with C; T381 is substituted with L; M437 is substituted with F; M437 is substituted with D; N425 is substituted with G; I20 is substituted with A; E649 is substituted with F; Q655 is substituted with N; D659 is substituted with E; G681 is substituted with P; W688 is substituted with Q; F761 is substituted with K; and K779 is substituted with A.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: M437 is substituted with F; I20 is substituted with A; G681 is substituted with P; and K779 is substituted with A.

In another embodiment, provided herein is a T7 RNA polymerase comprising the amino acid sequence set out in SEQ ID NO:1 or a functional fragment thereof, wherein the amino acid sequence comprises at least one feature selected from the group consisting of: M437 is substituted with F; I20 is substituted with A; G681 is substituted with P; and K779 is substituted with A.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises G681P substitution.

In another embodiment, provided herein is a T7 RNA polymerase comprising the amino acid sequence of SEQ ID NO:1 or a functional fragment thereof, wherein the amino acid sequence comprises G681P substitution.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises M437F.

In another embodiment, provided herein is a T7 RNA polymerase comprising the amino acid sequence set out in SEQ ID NO:1 or a functional fragment thereof, wherein the amino acid sequence comprises M437F.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises I20A.

In another embodiment, provided herein is a T7 RNA polymerase comprising the amino acid sequence set out in SEQ ID NO:1 or a functional fragment thereof, wherein the amino acid sequence comprises I20A.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises W688K substitution.

In another embodiment, provided herein is a T7 RNA polymerase comprising the amino acid sequence of SEQ ID NO:1 or a functional fragment thereof, wherein the amino acid sequence comprises W688K substitution.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises W688Q substitution.

In another embodiment, provided herein is a T7 RNA polymerase comprising the amino acid sequence set out in SEQ ID NO:1 or a functional fragment thereof, wherein the amino acid sequence comprises W688Q substitution.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A. In another embodiment, provided herein is a T7 RNA polymerase comprising the amino acid sequence of SEQ ID NO:1 or a functional fragment thereof, wherein the amino acid sequence comprises K779A substitution.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises N15A.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises I20N.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises Q655N.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises D659E.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises F761K.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises A264F.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises A268C.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises N425G.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises A264V.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises T381L.

In another embodiment, provided herein is a T7 RNA polymerase comprising the amino acid sequence of SEQ ID NO:1 or a functional fragment thereof, wherein at least two, at least three, at least four, at least five, at least six, at least ten, or at least twenty amino acids are substituted to a different amino acid to that found at the corresponding amino acid position in SEQ ID NO:1.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises Q64H substitution.

In another embodiment, provided herein is a T7 RNA polymerase of the present disclosure that is full length.

In one embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one amino acid substitution at positions K779, N15, I20, Q655, D659, W688, F761, A264, A268, N425, or T381.

In one embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises an amino acid substitution at position K779 and at least one other substitution. In one embodiment, in addition to the substitution at position K779, the T7 RNA polymerase of the present invention comprises 2, 3 or 4 other substitutions. In one embodiment, in addition to the substitution at position K779, the T7 RNA polymerase comprises at least one other substitution selected from positions N15, I20, Q655, D659, W688, F761, A264, A268, N425, and T381. In one embodiment, in addition to the substitution at position K779, the T7 RNA polymerase comprises at least one other substitution selected from positions N15, I20, Q655 and D659. In one embodiment, in addition to the substitution at position K779, the T7 RNA polymerase comprises at least one other substitution selected from positions W688 and F761. In one embodiment, in addition to the substitution at position K779, the T7 RNA polymerase comprises at least one other substitution selected from positions A264, A268 and N425. In one embodiment, in addition to the substitution at position K779, the T7 RNA polymerase comprises at least one other substitution selected from positions A264, N425, and T381.

In one embodiment, provided herein is a T7 RNA polymerase comprising the amino acid sequence of SEQ ID NO:1 or a functional fragment thereof, wherein the amino acid sequence comprises K779A substitution and at least one other substitution. In one embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution and at least one other substitution.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises: (a) K779A and at least one other amino acid substitution; (b) M437F and at least one other amino acid substitution; (c) I20A and at least one other amino acid substitution; (d) G681P and at least one other amino acid substitution; (e) N15A and at least one other amino acid substitution; (f) I20N and at least one other amino acid substitution; (g) Q655N and at least one other amino acid substitution; (h) D659E and at least one other amino acid substitution; (i) W688Q and at least one other amino acid substitution; (j) F761K and at least one other amino acid substitution; (k) A264F and at least one other amino acid substitution; (l) A264V and at least one other amino acid substitution; (m) A268C and at least one other amino acid substitution; (n) N425G and at least one other amino acid substitution; or (o) T381L and at least one other amino acid substitution. In one embodiment, K779A substitution and at least one amino acid substitution is selected from the group consisting of: N15 is substituted with A, I20 is substituted with N or A or K, E649 is substituted with F, Q64 is substituted with H, E69 is substituted with P, A257 is substituted with M, A264 is substituted with F or V, A268 is substituted with C, T381 is substituted with L, N425 is substituted with G, D427 is substituted with C, M437 is substituted with F or D, E649 is substituted with F or A, Q655 is substituted with N, D659 is substituted with T or E or S, G681 is substituted with L or P, W688 is substituted with K or T or R or V or G or Q or A, F761 is substituted with K, and I784 is substituted with M.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A, N15A, I20N, Q655N, and D659E substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A, W688Q, and F761K substitutions.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A, A264F, A268C, and N425G substitutions.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A, A264V, T381L, and N425G substitutions.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises (a) K779A, A264F, A268C, N425G; (b) K779A, A264V, T381L, N425G; or (c) a combination thereof.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution, and at least one other amino acid substitution selected from the group consisting of: 120N, 120A, and 120K.

In another embodiment, provided herein is a T7 RNA polymerase comprising the amino acid sequence of SEQ ID NO:1 or a functional fragment thereof, wherein the amino acid sequence comprises K779A substitution, and at least one other amino acid substitution selected from the group consisting of: 120N, 120A, and 120K.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A and E649F substitutions.

In another embodiment, provided herein is a T7 RNA polymerase comprising the amino acid sequence of SEQ ID NO:1 or a functional fragment thereof, wherein the amino acid sequence comprises K779A and E649F substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A and Q64H substitutions.

In another embodiment, provided herein is a T7 RNA polymerase comprising the amino acid sequence of SEQ ID NO:1 or a functional fragment thereof, wherein the amino acid sequence comprises K779A and Q64H substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A and E69P substitutions.

In another embodiment, provided herein is a T7 RNA polymerase comprising the amino acid sequence of SEQ ID NO:1 or a functional fragment thereof, wherein the amino acid sequence comprises K779A and E69P substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A and A257M substitutions.

In another embodiment, provided herein is a T7 RNA polymerase comprising the amino acid sequence of SEQ ID NO:1 or a functional fragment thereof, wherein the amino acid sequence comprises K779A and A257M substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A and D427C substitutions.

In another embodiment, provided herein is a T7 RNA polymerase comprising the amino acid sequence of SEQ ID NO:1 or a functional fragment thereof, wherein the amino acid sequence comprises K779A and D427C substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A and I784M substitutions.

In another embodiment, provided herein is a T7 RNA polymerase comprising the amino acid sequence of SEQ ID NO:1 or a functional fragment thereof, wherein the amino acid sequence comprises K779A and I784M substitutions.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution, and at least one other amino acid substitution selected from the group consisting of: M437F and M437D.

In another embodiment, provided herein is a T7 RNA polymerase comprising the amino acid sequence of SEQ ID NO:1 or a functional fragment thereof, wherein the amino acid sequence comprises K779A substitution, and at least one other amino acid substitution selected from the group consisting of: M437F and M437D.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution, and at least one other amino acid substitution selected from the group consisting of: E649F and E649A.

In another embodiment, provided herein is a T7 RNA polymerase comprising the amino acid sequence of SEQ ID NO:1 or a functional fragment thereof, wherein the amino acid sequence comprises K779A substitution, and at least one other amino acid substitution selected from the group consisting of: E649F and E649A.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution, and at least one other amino acid substitution selected from the group consisting of: D659T, D659E, and D659S.

In another embodiment, provided herein is a T7 RNA polymerase comprising the amino acid sequence of SEQ ID NO:1 or a functional fragment thereof, wherein the amino acid sequence comprises K779A substitution, and at least one other amino acid substitution selected from the group consisting of: D659T, D659E, and D659S.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution, and at least one other amino acid substitution selected from the group consisting of: G681L and G681P.

In another embodiment, provided herein is a T7 RNA polymerase comprising the amino acid sequence of SEQ ID NO:1 or a functional fragment thereof, wherein the amino acid sequence comprises K779A substitution, and at least one other amino acid substitution selected from the group consisting of: G681L and G681P.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises K779A substitution, and at least one other amino acid substitution selected from the group consisting of: W688K, W688T, W688R, W688V, W688G, W688Q, and W688A.

In another embodiment, provided herein is a T7 RNA polymerase comprising the amino acid sequence of SEQ ID NO:1 or a functional fragment thereof, wherein the amino acid sequence comprises K779A substitution, and at least one other amino acid substitution selected from the group consisting of: W688K, W688T, W688R, W688V, W688G, W688Q, and W688A.

In another embodiment, provided herein is a T7 RNA polymerase comprising an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, or SEQ ID NO:29.

In another embodiment, the T7 RNA polymerase comprises an N-terminal tag having the formula M-Hₓ (SEQ ID NO: 47), wherein M is a methionine residue and Hₓ represents a chain of x histidine residues, where x is a whole integer between 0 and 20. In certain embodiments, x is 0, 1, 2, 3, 4, 5, 6, 7, 8, 9, 10, 15 or 20. In a specific embodiment, x is 6.

In another embodiment, provided herein is a T7 RNA polymerase comprising an amino acid sequence that is at least 80%, 85%, 90%, 95%, 96%, 97%, 98%, or 99% identical to the amino acid sequence set out in SEQ ID NO: 2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

In another embodiment, provided herein is a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the amino acid sequence includes at least one feature selected from the group consisting of: N8 is substituted with A or T or Y or Q or S; I13 is substituted with A or L or V or G or M or N; Q57 is substituted with N or S or T or H; E62 is substituted with D or P; A250 is substituted with M or L or V or I or G; A257 is substituted with F or V or M or L or I or G; A261 is substituted with C or M or L or I or G; T374 is substituted with L or Y or N or Q or S; N418 is substituted with G or T or Y or Q or S; D420 is substituted with E or C; M430 is substituted with A or L or V or I or G or F or D; E642 is substituted with D or F; Q648 is substituted with N or T or Y or S; D652 is substituted with E or T; G674 is substituted with A or L or V or I or M or P; W681 is substituted with H or Y or F or K or Q; F754 is substituted with K or W or Y or H; K772 is substituted with R or A; and I777 is substituted with A or L or V or M or G.

In another embodiment, provided herein is a T7 RNA polymerase comprising the amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the amino acid sequence comprises at least one feature selected from the group consisting of: N8 is substituted with A, I13 is substituted with A, I13 is substituted with N, Q57 is substituted with H, E62 is substituted with P, A250 is substituted with M, A257 is substituted with F, A257 is substituted with V, A261 is substituted with C, T374 is substituted with L, N418 is substituted with G, D420 is substituted with C, M430 is substituted with F, M430 is substituted with D, E642 is substituted with F, Q648 is substituted with N, D652 is substituted with T, D652 is substituted with E, G674 is substituted with P, W681 is substituted with K, W681 is substituted with Q, F754 is substituted with K, K772 is substituted with A, and I777 is substituted with M.

In some embodiments, a single point mutation in the T7 RNA polymerase variant can increase transcription efficiency in an in vitro transcription reaction (IVT) by between about 1.1-fold and about 10-fold, by between about 1.2-fold and about 7-fold, by between about 1.3-fold and about 5-fold, by between about 1.5-fold and about 2.5-fold or by between about 1.5-fold and about 6-fold or by at least 2-fold compared to the rate of a wild type T7 RNA polymerase or a T7 RNA polymerase that lacks the point mutation. The positive effect of single point mutations can multiply when combined as multiple point mutations in a T7 RNA polymerase. In some embodiments, the T7 RNA polymerase variant can contain at least 2, 3, 4, 5, 6, 7, 8, 9, 10, 11, 12, 13, 14, 15, 16, 17, 18, 19 or 20 amino acid substitutions. In some embodiments, transcription efficiency is determined by analyzing the RNA concentration synthesized during each IVT reaction. In some embodiments, use of an RNA polymerase variant increases the transcription efficiency (e.g., RNA yield and/or rate of transcription) by at least 10%, at least 20%, at least 30%, at least 40%, at least 50%, at least 60%, at least 70%, at least 80%, or at least 90%. In some embodiments, the control RNA polymerase is a wild-type RNA polymerase comprising the amino acid sequence of SEQ ID NO: 2 (“wild-type T7 RNA polymerase”). In other embodiment, the control RNA polymerase is a wild-type RNA polymerase modified to include a N-terminal M-Polyhistidine tag (SEQ ID NO: 1).

In some embodiments, use of the T7 RNA polymerase variants of the present disclosure, for example, in an in vitro transcription reaction, improves fidelity (e.g., reduces mutation rate) of transcription. For example, use of the T7 RNA polymerase variant may improve fidelity of transcription by at least 5%, at least 10%, or at least 20%. In some embodiments, use of the T7 RNA polymerase variant improves fidelity of transcription by at least 30%, at least 40%, at least 50%, at least 60%, at least 70%, at least 80%, at least 90%, or at least 100%. In some embodiments, use of the T7 RNA polymerase variant improves fidelity of transcription by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. The T7 RNA polymerase variants of the present disclosure that improve fidelity of transcription will produce RNA transcript (e.g., mRNA transcript) with a lower rate or total number of mutations than a control T7 RNA polymerase. In some embodiments, the control T7 RNA polymerase is a wild-type T7 RNA polymerase comprising the amino acid sequence of SEQ ID NO: 2 (“wild-type T7 RNA polymerase”). In other embodiment, the control RNA polymerase is a wild-type RNA polymerase modified to include a N-terminal M-Polyhistidine tag (SEQ ID NO: 1).

In some embodiments, use of the T7 RNA polymerase variants of the present disclosure, for example, in an in vitro transcription reaction, increases % RNA integrity. In some embodiments, use of the T7 RNA polymerase variant increases % RNA integrity by at least 2%, at least 5%, at least 10%, at least 20%, at least 30%, at least 40%, at least 50%, at least 60%, at least 70%, at least 80%, at least 90%, or at least 100%. In some embodiments, the % RNA integrity of the RNA transcripts made using the T7 RNA polymerase variants of the present disclosure is at least 60%. In some embodiments, use of the T7 RNA polymerase variants of the present disclosure, for example, in an in vitro transcription reaction, reduces the amount of double-stranded RNA (dsRNA) contamination in the in vitro transcription reaction. For example, use of the T7 RNA polymerase variant may reduce the amount of dsRNA contamination in the in vitro transcription reaction by at least 5%, at least 10%, or at least 20%. In some embodiments, use of the T7 RNA polymerase variant reduces the amount of dsRNA contamination in the in vitro transcription reaction by at least 30%, at least 40%, at least 50%, at least 60%, at least 70%, at least 80%, at least 90%, or at least 100%. In some embodiments, use of the T7 RNA polymerase variant reduces the amount of dsRNA contamination in the in vitro transcription reaction by 20-100%, 20-90%, 20-80%, 20-70%, 20-60%, 20-50%, 30-100%, 30-90%, 30-80%, 30-70%, 30-60%, 30-50%, 40-100%, 40-90%, 40-80%, 40-70%, 40-60%, 40-50%, 50-100%, 50-90%, 50-80%, 50-70%, or 50-60%. In some embodiments, the control T7 RNA polymerase is a wild-type T7 RNA polymerase comprising the amino acid sequence of SEQ ID NO: 2 (“wild-type T7 RNA polymerase”). In other embodiment, the control RNA polymerase is a wild-type RNA polymerase modified to include a N-terminal M-Polyhistidine tag (SEQ ID NO: 1).

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1; and wherein the T7 RNA polymerase has improved enzyme property over the T7 RNA polymerase having the amino acid sequence as set out in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1; and wherein the T7 RNA polymerase has improved transcription efficiency in an in vitro transcription reaction (IVT) by between about 1.1-fold and about 10-fold, by between about 1.2-fold and about 7-fold, by between about 1.3-fold and about 5-fold, by between about 1.5-fold and about 2.5-fold or by between about 1.5-fold and about 6-fold or by at least 2-fold compared to the T7 RNA polymerase having the amino acid sequence as set out in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1; and wherein the T7 RNA polymerase shows improved fidelity of transcription by at least 5%, at least 10%, or at least 20% compared to the T7 RNA polymerase having the amino acid sequence as set out in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1; and wherein the T7 RNA polymerase shows increased % RNA integrity in an in vitro transcription reaction compared to the T7 RNA polymerase having the amino acid sequence as set out in SEQ ID NO:1.

In one embodiment there is provided a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1; and wherein the T7 RNA polymerase reduced amount of double-stranded RNA (dsRNA) contamination in the in vitro transcription reaction by at least 5%, at least 10%, or at least 20% compared to the T7 RNA polymerase having the amino acid sequence as set out in SEQ ID NO:1.

In another aspect, the present invention provides a composition comprising a T7 RNA polymerase of the present invention. Thus, in some embodiments, provided herein is a composition comprising a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1.

In some embodiments, provided herein is a composition comprising a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G.

In some embodiments, provided herein is a composition comprising a T7 RNA polymerase comprising the amino acid sequence of SEQ ID NO:1 or a functional fragment thereof, wherein the amino acid sequence comprises at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M.

In some embodiments, provided herein is a composition comprising a T7 RNA polymerase comprising the amino acid sequence of SEQ ID NO:1 or a functional fragment thereof, wherein the amino acid sequence comprises at least one feature selected from the group consisting of: Q64 is substituted with H, E69 is substituted with P, N15 is substituted with A, I20 is substituted with N, I20 is substituted with A, A264 is substituted with F, A264 is substituted with V, A268 is substituted with C, T381 is substituted with L, M437 is substituted with F, M437 is substituted with D, N425 is substituted with G, E649 is substituted with F, Q655 is substituted with N, D659 is substituted with E, G681 is substituted with P, W688 is substituted with Q, F761 is substituted with K, and K779 is substituted with A.

In some embodiments, provided herein is a composition comprising a T7 RNA polymerase comprising the amino acid sequence of SEQ ID NO:1 or a functional fragment thereof, wherein the amino acid sequence comprises at least one feature selected from the group consisting of: G681 is substituted with P; M437 is substituted with F; I20 is substituted with A; and K779 is substituted with A.

In some embodiments, provided herein is a composition comprising a T7 RNA polymerase comprising the amino acid sequence of SEQ ID NO:1 or a functional fragment thereof, wherein at least two, at least three, at least four, at least five, at least six, at least ten, or at least twenty amino acids are substituted to a different amino acid to that found at the corresponding amino acid position in SEQ ID NO:1.

In some embodiments, provided herein is a composition comprising a T7 RNA polymerase comprising the amino acid sequence of SEQ ID NO:1 or a functional fragment thereof, wherein the amino acid substitution comprises (a) K779A and at least one other amino acid substitution; (b) M437F and at least one other amino acid substitution; (c) I20A and at least one other amino acid substitution; (d) G681P and at least one other amino acid substitution; (e) N15A and at least one other amino acid substitution; (f) I20N and at least one other amino acid substitution; (g) Q655N and at least one other amino acid substitution; (h) D659E and at least one other amino acid substitution; (i) W688Q and at least one other amino acid substitution; (j) F761K and at least one other amino acid substitution; (k) A264F and at least one other amino acid substitution; (l) A264V and at least one other amino acid substitution; (m) A268C and at least one other amino acid substitution; (n) N425G and at least one other amino acid substitution; or (o) T381L and at least one other amino acid substitution.

In one embodiment, K779A substitution and at least one other substitution selected from the group consisting of: N15 is substituted with A, I20 is substituted with N or A or K, E649 is substituted with F, Q64 is substituted with H, E69 is substituted with P, A257 is substituted with M, A264 is substituted with F or V, A268 is substituted with C, T381 is substituted with L, N425 is substituted with G, D427 is substituted with C, M437 is substituted with F or D, E649 is substituted with F or A, Q655 is substituted with N, D659 is substituted with T or E or S, G681 is substituted with L or P, W688 is substituted with K or T or R or V or G or Q or A, F761 is substituted with K, and I784 is substituted with M.

In some embodiments, provided herein is a composition comprising a T7 RNA polymerase comprising an amino acid sequence selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14 SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28; or SEQ ID NO:29.

In some embodiments, provided herein is a composition comprising a polynucleotide, wherein the polynucleotide comprises a nucleic acid sequence selected from the group consisting of: SEQ ID NO: 30, SEQ ID NO:31, SEQ ID NO:32, SEQ ID NO: 33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO: 44, SEQ ID NO:45, or SEQ ID NO:46, wherein the polynucleotide encodes the T7 RNA polymerase as described herein.

In some embodiments, provided herein is a composition comprising a T7 RNA polymerase, wherein the T7 RNA polymerase comprises an N-terminal tag having the formula M-Hₓ (SEQ ID NO: 47), wherein M is a methionine residue and Hₓ represents a chain of x histidine residues, where x is a whole integer between 0 and 20. In certain embodiments, x is 0, 1, 2, 3, 4, 5, 6, 7, 8, 9, 10, 15 or 20. In a specific embodiment, x is 6.

In some embodiments, provided herein is a composition comprising a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

In some embodiments, provided herein is a composition comprising a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes at least one feature selected from the group consisting of: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G.

In some embodiments, provided herein is a composition comprising a T7 RNA polymerase as described herein.

In some embodiments, provided herein is a kit comprising an in vitro transcription component and a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1. The in vitro transcription component can be, for example, a transcription reagent, a polynucleotide template, nucleoside triphosphates, and/or a cap analog.

In some embodiments, provided herein is a kit comprising an in vitro transcription component and a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G. The in vitro transcription component can be a transcription reagent, a polynucleotide template, nucleoside triphosphates, and a cap analog.

In some embodiments, provided herein is a kit comprising an in vitro transcription component and a T7 RNA polymerase comprising the amino acid sequence of SEQ ID NO:1 or a functional fragment thereof, wherein the amino acid sequence comprises at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M. The in vitro transcription component can be a transcription reagent, a polynucleotide template, nucleoside triphosphates, and a cap analog.

In some embodiments, provided herein is a kit comprising an in vitro transcription component and a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

In some embodiments, provided herein is a kit comprising an in vitro transcription component and a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the T7 RNA polymerase amino acid sequence includes at least one feature selected from the group consisting of: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G. In some embodiments, provided herein is a kit comprising an in vitro transcription component and a T7 RNA polymerase as described herein.

In some embodiments, provided herein is a kit comprising an in vitro transcription component and a T7 RNA polymerase as described herein, wherein the in vitro transcription component is selected from the group consisting of: a transcription reagent, a deoxyribonucleic acid (DNA), nucleoside triphosphates, and a cap analog.

### Nucleic Acids

In another aspect, provided herein are polynucleotides encoding the T7 RNA polymerase of the present disclosure. In some embodiments, the polynucleotide encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO: 1.

In some embodiments, the polynucleotide encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the amino acid sequence comprises at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G.

In some embodiments, the polynucleotide encodes a T7 RNA polymerase comprising the amino acid sequence of SEQ ID NO:1 or a functional fragment thereof, wherein the amino acid sequence comprises at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M.

In some embodiments, the polynucleotide encodes a T7 RNA polymerase comprising the amino acid sequence of SEQ ID NO:1 or a functional fragment thereof, wherein the amino acid sequence comprises at least one feature selected from the group consisting of: Q64 is substituted with H, E69 is substituted with P, N15 is substituted with A, I20 is substituted with N, I20 is substituted with A, A264 is substituted with F, A264 is substituted with V, A268 is substituted with C, T381 is substituted with L, M437 is substituted with F, M437 is substituted with D, N425 is substituted with G, E649 is substituted with F, Q655 is substituted with N, D659 is substituted with E, G681 is substituted with P, W688 is substituted with Q, F761 is substituted with K, and K779 is substituted with A.

In some embodiments, the polynucleotide encodes a T7 RNA polymerase comprising the amino acid sequence of SEQ ID NO:1 or a functional fragment thereof, wherein the amino acid sequence comprises at least one feature selected from the group consisting of: M437 is substituted with F; I20 is substituted with A; G681 is substituted with P; and K779 is substituted with A.

In some embodiments, the polynucleotide encodes a T7 RNA polymerase comprising the amino acid sequence of SEQ ID NO:1 or a functional fragment thereof, wherein K779 is substituted with A.

In some embodiments, the polynucleotide encodes a T7 RNA polymerase comprising the amino acid sequence of SEQ ID NO:1 or a functional fragment thereof, wherein at least two, at least three, at least four, at least five, at least six, at least seven, at least eight, at least nine, at least 10, or at least 20 amino acids are substituted to a different amino acid to that found at the corresponding amino acid position in SEQ ID NO:1.

In some embodiments, the polynucleotide encodes a T7 RNA polymerase comprising the amino acid sequence of SEQ ID NO:1 or a functional fragment thereof, wherein the amino acid substitution comprises: (a) K779A and at least one other amino acid substitution; (b) M437F and at least one other amino acid substitution; (c) 120A and at least one other amino acid substitution; (d) G681P and at least one other amino acid substitution; (e) N15A and at least one other amino acid substitution; (f) 120N and at least one other amino acid substitution; (g) Q655N and at least one other amino acid substitution; (h) D659E and at least one other amino acid substitution; (i) W688Q and at least one other amino acid substitution; (j) F761K and at least one other amino acid substitution; (k) A264F and at least one other amino acid substitution; (l) A264V and at least one other amino acid substitution; (m) A268C and at least one other amino acid substitution; (n) N425G and at least one other amino acid substitution; or (o) T381L and at least one other amino acid substitution.

In some embodiments, the polynucleotide encodes a T7 RNA polymerase comprising the amino acid sequence of SEQ ID NO:1 or a functional fragment thereof, wherein the T7 RNA polymerase comprises K779A substitution and at least one other amino acid substitution selected from the group consisting of: N15 is substituted with A, I20 is substituted with N or A or K, E649 is substituted with F, Q64 is substituted with H, E69 is substituted with P, A257 is substituted with M, A264 is substituted with F or V, A268 is substituted with C, T381 is substituted with L, N425 is substituted with G, D427 is substituted with C, M437 is substituted with F or D, E649 is substituted with F or A, Q655 is substituted with N, D659 is substituted with T or E or S, G681 is substituted with L or P, W688 is substituted with K or T or R or V or G or Q or A, F761 is substituted with K, and I784 is substituted with M.

In some embodiments, the polynucleotide encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

In some embodiments, the polynucleotide encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes at least one feature selected from the group consisting of: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G.

In some embodiments, the polynucleotide comprises a nucleic acid sequence selected from the group consisting of SEQ ID NO: 30, SEQ ID NO:31, SEQ ID NO:32, SEQ ID NO: 33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO: 44, SEQ ID NO:45, or SEQ ID NO:46.

In some embodiments, provided herein is a polynucleotide that encodes a T7 RNA polymerase as described herein.

In some embodiments, provided herein is a vector comprising the polynucleotides encoding the T7 RNA polymerase variants of the present disclosure. In some embodiments, the vector comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 1 or a functional fragment thereof, wherein the T7 RNA polymerase amino acid sequence includes the feature that: at least one amino acid selected from the group consisting of amino acid, N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO: 1.

In some embodiments, provided herein is a vector that comprises a polynucleotide that encodes a T7 RNA polymerase comprising the amino acid sequence of SEQ ID NO:1 or a functional fragment thereof, wherein the amino acid sequence comprises at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G.

In some embodiments, provided herein is a vector that comprises a polynucleotide that encodes a T7 RNA polymerase comprising the amino acid sequence of SEQ ID NO:1 or a functional fragment thereof, wherein the amino acid sequence comprises at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M.

In some embodiments, provided herein is a vector that comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

In some embodiments, provided herein is a vector that comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes at least one feature selected from the group consisting of: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G.

In some embodiments, provided herein is a vector that comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes at least one feature selected from the group consisting of: N8 is substituted with A, I13 is substituted with A, I13 is substituted with N, Q57 is substituted with H, E62 is substituted with P, A250 is substituted with M, A257 is substituted with F, A257 is substituted with V, A261 is substituted with C, T374 is substituted with L, N418 is substituted with G, D420 is substituted with C, M430 is substituted with F, M430 is substituted with D, E642 is substituted with F, Q648 is substituted with N, D652 is substituted with T, D652 is substituted with E, G674 is substituted with P, W681 is substituted with K, W681 is substituted with Q, F754 is substituted with K, K772 is substituted with A, and I777 is substituted with M.

In some embodiments, provided herein is a vector that comprises a polynucleotide that encodes a T7 RNA polymerase as described herein.

### Host Cells

In another aspect, provided herein is a host cell comprising a T7 RNA polymerase, wherein the T7 RNA polymerase comprises at least one amino acid substitution. In another embodiment, the host cell is E. coli. In some embodiments, the host cell comprises two or more T7 RNA polymerase variants.

In another aspect, provided herein is a host cell comprising a polynucleotide provided herein (e.g., encoding the T7 RNA polymerase variant provided herein). In some embodiments, the host cell comprises two or more polynucleotides provided herein (e.g., 2, 3, 4, 5, 6, 7, 8, 9, 10 or more nucleic acids).

In some embodiments, provided herein is a host cell comprising a vector comprising the polynucleotide that encode the T7 RNA polymerase variants of the present disclosure. In some embodiments, the host cell comprises a vector which comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid, N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1.

In some embodiments, provided herein is a host cell comprising a vector which comprises a polynucleotide that encodes a T7 RNA polymerase comprising the amino acid sequence of SEQ ID NO:1 or a functional fragment thereof, wherein the amino acid sequence comprises at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G.

In some embodiments, provided herein is a host cell comprising a vector which comprises a polynucleotide that encodes a T7 RNA polymerase comprising the amino acid sequence of SEQ ID NO:1 or a functional fragment thereof, wherein the amino acid sequence comprises at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M.

In some embodiments, provided herein is a host cell comprising a vector which comprises a polynucleotide that encodes a T7 RNA polymerase comprising the amino acid sequence of SEQ ID NO:1 or a functional fragment thereof, wherein at least two, at least three, at least four, at least five, at least six, at least seven, at least eight, at least nine, at least 10, or at least 20 amino acids are substituted to a different amino acid to that found at the corresponding amino acid position in SEQ ID NO:1.

In some embodiments, provided herein is a host cell comprising a vector which comprises a polynucleotide that encodes a T7 RNA polymerase comprising the amino acid sequence of SEQ ID NO:1 or a functional fragment thereof, wherein the amino acid substitution comprises K779A.

In some embodiments, provided herein is a host cell comprising a vector which comprises a polynucleotide that encodes a T7 RNA polymerase comprising the amino acid sequence of SEQ ID NO:1 or a functional fragment thereof, wherein the T7 RNA polymerase further comprises: (a) K779A and at least one other amino acid substitution; (b) M437F and at least one other amino acid substitution; (c) I20A and at least one other amino acid substitution; (d) G681P and at least one other amino acid substitution; (e) N15A and at least one other amino acid substitution; (f) I20N and at least one other amino acid substitution; (g) Q655N and at least one other amino acid substitution; (h) D659E and at least one other amino acid substitution; (i) W688Q and at least one other amino acid substitution; (j) F761K and at least one other amino acid substitution; (k) A264F and at least one other amino acid substitution; (l) A264V and at least one other amino acid substitution; (m) A268C and at least one other amino acid substitution; (n) N425G and at least one other amino acid substitution; or (o) T381L and at least one other amino acid substitution. In one embodiment, K779A substitution and at least one other amino acid substitution selected from the group consisting of: N15 is substituted with A, I20 is substituted with N or A or K, E649 is substituted with F, Q64 is substituted with H, E69 is substituted with P, A257 is substituted with M, A264 is substituted with F or V, A268 is substituted with C, T381 is substituted with L, N425 is substituted with G, D427 is substituted with C, M437 is substituted with F or D, E649 is substituted with F or A, Q655 is substituted with N, D659 is substituted with T or E or S, G681 is substituted with L or P, W688 is substituted with K or T or R or V or G or Q or A, F761 is substituted with K, and I784 is substituted with M.

In some embodiments, provided herein is a host cell comprising a vector which comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

In some embodiments, provided herein is a host cell comprising a vector which comprises a polynucleotide that encodes a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO: 2 or a functional fragment thereof, wherein the T7 RNA polymerase amino acid sequence includes at least one feature selected from the group consisting of: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G.

In some embodiments, provided herein is a host cell comprising a vector that comprises a polynucleotide that encodes a T7 RNA polymerase as described herein.

Methods of Performing in vitro Transcription

In some embodiments, provided herein are methods of performing an in vitro transcription (IVT) reaction using the T7 RNA polymerase of the present disclosure. Methods of performing IVT reactions are known to a skilled person. In some embodiments, provided herein is a method of performing an IVT reaction comprising combining a deoxyribonucleic acid (DNA) and nucleoside triphosphates (e.g., modified or unmodified ATP, modified or unmodified UTP, modified or unmodified GTP, and/or modified or unmodified CTP) with a T7 ribonucleic acid (RNA) polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to SEQ ID NO:1 or a functional fragment thereof, wherein the amino acid sequence includes at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to SEQ ID NO:1 or a functional fragment thereof, wherein the amino acid sequence includes at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to SEQ ID NO:1 or a functional fragment thereof, wherein the amino acid sequence includes at least one feature selected from the group consisting of: Q64 is substituted with H, E69 is substituted with P, N15 is substituted with A, I20 is substituted with N, I20 is substituted with A, A264 is substituted with F, A264 is substituted with V, A268 is substituted with C, T381 is substituted with L, M437 is substituted with F, M437 is substituted with D, N425 is substituted with G, E649 is substituted with F, Q655 is substituted with N, D659 is substituted with E, G681 is substituted with P, W688 is substituted with Q, F761 is substituted with K, and K779 is substituted with A. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to SEQ ID NO:1 or a functional fragment thereof, wherein the amino acid sequence includes at least one feature selected from the group consisting of: M437 is substituted with F; I20 is substituted with A, G681 is substituted with P; and K779 is substituted with A. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to SEQ ID NO:1 or a functional fragment thereof, wherein K779 is substituted with A. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising the amino acid sequence of SEQ ID NO:1 or a functional fragment thereof, wherein at least two, at least three, at least four, at least five, at least six, at least seven, at least eight, at least nine, at least 10, or at least 20 amino acids are substituted to a different amino acid to that found at the corresponding amino acid position in SEQ ID NO:1. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising the amino acid sequence of SEQ ID NO:1 or a functional fragment thereof, wherein the amino acid substitution comprises: (a) K779A and at least one other amino acid substitution; (b) M437F and at least one other amino acid substitution; (c) I20A and at least one other amino acid substitution; (d) G681P and at least one other amino acid substitution; (e) N15A and at least one other amino acid substitution; (f) I20N and at least one other amino acid substitution; (g) Q655N and at least one other amino acid substitution; (h) D659E and at least one other amino acid substitution; (i) W688Q and at least one other amino acid substitution; (j) F761K and at least one other amino acid substitution; (k) A264F and at least one other amino acid substitution; (l) A264V and at least one other amino acid substitution; (m) A268C and at least one other amino acid substitution; (n) N425G and at least one other amino acid substitution; or (o) T381L and at least one other amino acid substitution. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising the amino acid sequence of SEQ ID NO:1 or a functional fragment thereof, wherein the T7 RNA polymerase comprises K779A and at least one other amino acid substitution selected from the group consisting of: N15 is substituted with A, I20 is substituted with N or A or K, E649 is substituted with F, Q64 is substituted with H, E69 is substituted with P, A257 is substituted with M, A264 is substituted with F or V, A268 is substituted with C, T381 is substituted with L, N425 is substituted with G, D427 is substituted with C, M437 is substituted with F or D, E649 is substituted with F or A, Q655 is substituted with N, D659 is substituted with T or E or S, G681 is substituted with L or P, W688 is substituted with K or T or R or V or G or Q or A, F761 is substituted with K, and I784 is substituted with M. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the T7 RNA polymerase amino acid sequence includes the feature that: at least one amino acid selected from the group consisting of amino acid N8, 113, Q57, E62, A250, A257, A261, T374, N418, D420, M430, E642, Q648, D652, G674, W681, F754, K772, and I777 is substituted to a different amino acid to that found at the corresponding position in SEQ ID NO: 2. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the T7 RNA polymerase amino acid sequence includes the feature that: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates (e.g., modified or unmodified ATP, modified or unmodified UTP, modified or unmodified GTP, and/or modified or unmodified CTP) with a T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the T7 RNA polymerase amino acid sequence includes the feature that: N8 is substituted with A, I13 is substituted with A, 113 is substituted with N, Q57 is substituted with H, E62 is substituted with P, A250 is substituted with M, A257 is substituted with F, A257 is substituted with V, A261 is substituted with C, T374 is substituted with L, N418 is substituted with G, D420 is substituted with C, M430 is substituted with F, M430 is substituted with D, E642 is substituted with F, Q648 is substituted with N, D652 is substituted with T, D652 is substituted with E, G674 is substituted with P, W681 is substituted with K, W681 is substituted with Q, F754 is substituted with K, K772 is substituted with A, and I777 is substituted with M. In some embodiments, the nucleoside triphosphates comprise modified nucleoside triphosphates. In some embodiments, the modified nucleoside triphosphates comprise a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, modified nucleobase is 1-methylpseudouridine.

In some embodiments, provided herein is a method of performing an IVT reaction comprising combining a DNA and nucleoside triphosphates with a T7 RNA polymerase as described herein.

The present invention also provides a T7 RNA polymerase of the present invention for use in an IVT reaction. The IVT reaction may comprise combining a deoxyribonucleic acid (DNA) and nucleoside triphosphates (e.g., modified or unmodified ATP, modified or unmodified UTP, modified or unmodified GTP, and/or modified or unmodified CTP) with the T7 RNA polymerase of the present invention.

Thus in an embodiment the present invention provides a T7 ribonucleic acid (RNA) polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1, for use in performing an IVT reaction.

Modified nucleosides may include modified nucleobases. For example, an IVT reaction of the present disclosure may be carried out in the presence of a modified nucleobase selected from pseudouridine, 1-methylpseudouridine, 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, the IVT reaction is carried out in the presence of a combination of at least two (e.g., 2, 3, 4 or more) of the foregoing modified nucleobases. In other embodiments the IVT reaction is carried out in the presence of modified and unmodified ATP; modified and unmodified UTP; modified and unmodified GTP; and/or modified and unmodified CTP.

The T7 RNA polymerase variants and methods provided herein of using the T7 RNA polymerase variants are of particular commercial importance and relevance, as they substantially increase the RNA yield and quality. Large RNAs are not compatible with traditional methods used to purge undesired species from the in vitro transcription reactions of RNA production (HPLC purification, cellulose column, etc). Therefore, reducing the formation of such species is necessary in order to improve product quality. This is a significant clinical advantage for platforms that use T7 RNA polymerase, as it significantly lowers the cost of manufacturing for RNA manufacturing (e.g., mRNA manufacturing) and improves the quality of material going into the clinic (improved reactogenicity/toxicity profile, reduced patient risk, etc).

### EXAMPLES

Many modifications and variations of the present disclosure are possible in light of the above teachings. It is therefore to be understood that, within the scope of the appended claims, a skilled person in the art would recognize that the invention may be practiced otherwise than as specifically described. The illustrative embodiments and examples should not be construed as limiting the invention.

### Example 1: Construction of T7 RNA Polymerase Expression Vectors

The wild type (WT) T7 RNA polymerase gene was amplified from E. coli BL21 cells (NEB). A synthetic gene of this WT T7 RNA polymerase gene containing a 6-histidine tag (SEQ ID NO: 48) was constructed and subcloned into a suitable E. coli expression vector. The plasmid construct was transformed into an E. coli expression strain, and T7 RNA polymerase gene sequence was confirmed by Sanger sequencing. Suitable expression vectors and/or strains are known to a skilled person. Vectors encoding T7 RNA polymerase variants were prepared according to the same methods.

### Example 2: High-Throughput (HTP) Growth, Expression and Purification of T7 RNA Polymerase and Variants

Transformed E. coli cells were selected by plating onto LB agar plates containing 1% glucose and 30 μg/mL chloramphenicol. After overnight incubation at 37° C., colonies were plated into the wells of 96-well shallow flat bottom NUNC™ (Thermo-Scientific) plates filled with 180 μL/well LB medium supplemented with 1% glucose and 30 μg/mL chloramphenicol. The cultures were allowed to grow overnight for 18-20 hours in an incubated, humidity-controlled shaker (200 rpm, 30° C. and 85% relative humidity; Kuhner). 20 μL of overnight growth samples were transferred into Coster 96-well deep well plates filled with 380 μL of Terrific Broth supplemented with 30 μg/mL chloramphenicol. The plates were incubated I20 minutes in an incubated, humidity-controlled shaker (250 rpm, 30° C. and 85% relative humidity; Kuhner) until measured to have an average OD₆₀₀ between 0.4-0.8 via a Spectromax M2 microplate reader. The cells were then induced with 40 μL of 10 mM IPTG in sterile water and incubated overnight for 18-20 hours in an incubated, humidity-controlled shaker (250 rpm, 30° C. and 85% relative humidity; Kuhner). The cells were pelleted (4000 rpm×20 min), the supernatants were discarded, and the cells were frozen at −80° C. prior to lysis and analysis.

Lysis was conducted by resuspending cells in 400 μL of lysis buffer (10 mM Tris·HCl pH 7.5, 1 mg/mL lysozyme, 500 mM NaCl and 0.5 mg/mL polymyxin B sulfate) and the mixture was agitated for 2 hours at room temperature. Lysates were then pelleted (4000 rpm×10 min) and the clarified supernatants were purified by metal-affinity chromatography using HisPur™ Ni-NTA spin plates (Thermo Fisher). Plates were equilibrated twice with 200 μL of T7 RNA Polymerase (T7RNAP) purification buffer (10 mM Tris·HCl pH 7.5, 20 mM Imidazole, 500 mM NaCl) per well and centrifuged (1000 rcf×2 min) at 4° C. Purification plates were then loaded with 300 μL of clarified cell lysate and were incubated for 30 minutes with moderate agitation on a benchtop plate shaker (500 rpm). Lysate was then eluted by centrifugation (1000 rcf×2 min) and were washed three times with 200 μL of T7RNAP wash buffer (10 mM Tris·HCl pH 7.5, 20 mM Imidazole, 500 mM NaCl) followed by centrifugation (1000 rcf×2 min) at 4° C. The purified enzyme was eluted by adding 100 μL of T7RNAP elution buffer (10 mM Tris·HCl pH 7.5, 250 mM Imidazole, 250 mM NaCl) to each well, gently agitating at room temperature for 5 minutes, and centrifuging (1000 rcf×2 min) to collect the eluate.

Eluates were buffer exchanged using Zeba™ 40 kDa cutoff Spin desalting plates (Thermo Fisher). Plates were equilibrated twice with 375 μL of 2×T7RNAP storage buffer (100 mM Tris·HCl pH 8.0, 200 mM NaCl, 2 mM DTT, 2 mM EDTA) per well and centrifuged (1100 rcf×2 min) at 4° C. Desalting plates were then loaded with 90 μL of the HisPur™ Ni-NTA spin plate eluate and centrifuged (1100 rcf×2 min) at 4° C. The eluate from the desalting plate was retained and mixed with an equal volume of glycerol for a final storage buffer concentration of 50 mM Tris·HCl pH 8.0, 100 mM NaCl, 1 mM DTT, 1 mM EDTA and 50% v/v glycerol.

### Example 3: High-Throughput IVT Reactions of T7 RNA Polymerase and Variants and Analysis by Capillary Gel Electrophoresis

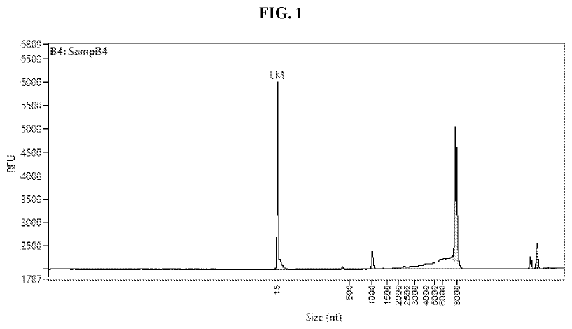

96 well PCR plates were charged with 9.5 μL of reaction Master Mix (42 mM Tris·HCl pH 8.0, 25.3 mM MgCl2, 6.3 mM each NTP (ATP, CTP, GTP, UTP), 10.5 mM DTT, 2.1 mM spermidine, 52.6 ng/μL linearized sample DNA (9,232 nucleotides in length) to serve as template for the RNA polymerase reaction, 0.002 U/μL yeast inorganic pyrophosphatase (NEB), 1.05 U/μL Rnase Inhibitor (NEB)). Reactions were initiated by the addition of 0.5 μL of desalted, purified enzyme followed by incubation at 30° C. for 2 hours. Reactions were then quenched by the addition of 90 μL of Tris-EDTA (TE) pH 8.0. After mixing, 20 μL of quenched material was diluted again into 180 μL of TE pH 8.0, and then analyzed on an Agilent Fragment Analyzer 5300 system equipped with a 96 well capillary head according to the Fragment Analyzer's integrated extended RNA analysis method. The overall yield of full-length RNA was assessed by monitoring the integrated intensity of the sample peak between 8000 and 10000 nt in length, while RNA integrity was assessed by normalizing this peak to the integrated intensity of sample peaks between 100 and 8000 nt in length (example data for the wild type T7 RNA polymerase is shown in FIG. 1.).

### Example 4: High-Throughput IVT Reactions of T7 RNA Polymerase and Variants and Analysis by Anti-polyA Molecular Beacon

384 well PCR plates (Bio-Rad) were charged with 950 nL of reaction Master Mix (42 mM Tris·HCl pH 8.0, 25.3 mM MgCl2, 6.3 mM each NTP, 10.5 mM DTT, 2.1 mM spermidine, 52.6 ng/μL linearized sample DNA (9,232 nucleotides in length), 0.002 U/μL yeast inorganic pyrophosphatase (NEB), 1.05 U/μL Rnase Inhibitor (NEB)). Reactions were initiated by the addition of 50 nL of desalted, purified T7 RNA polymerase (WT or variants) followed by incubation at 30° C. for 2 hours. Reactions were then quenched by the addition of 4 μL of Tris-EDTA (TE) pH 8.0 containing 1 μM anti-polyA molecular beacon (Sigma Aldrich) and transferred to a Bio-Rad CFX384 q-PCR instrument. Following a 5 minute incubation at 30° C., total FAM fluorescence was measured as an endpoint reporter assay for full length RNA transcription activity.

Based on the results of Examples 3 and 4, the highest performing mutants based on RNA integrity, RNA yield, and Molecular Beacon signal were progressed to scale-up testing.

### Example 5: Testing the Efficiency of Twelve T7 RNA Polymerase Variants

Initially, twelve T7 RNA polymerase variants were prepared using the methods known to a person skilled in the art (see e.g., Example 2). The highest performing T7 RNA polymerase variants (based on Examples 3 and 4) were tested in IVT reactions along with the wild-type T7. All IVT reactions were carried out according to standard methods using components listed in Table 3 with a final volume of 100 μL per reaction. Reactions were initiated by the addition of a linearized DNA template followed by incubation at 30° C. for 5 hours. Reactions were then quenched by the addition of Lithium chloride (LiCl) and storing the reactions at −20° C. to precipitate synthesized RNA. Following LiCl precipitation, RNAs were rehydrated in nuclease-free water and quantified for total RNA using Nanodrop spectrophotometry (Thermo Fisher Scientific).

TABLE 3
List of IVT reaction components

| Reagent | Vendor |
|---|---|
| Nuclease free water | — |
| Tris-HCl pH 7.0 (1M) | Sigma-Aldrich |
| Magnesium chloride (2M) | Sigma-Aldrich |
| Adenosine-5′-triphosphate (100 mM) | NEB |
| Cytosine-5′-triphosphate (100 mM) | NEB |
| Guanosine-5′-triphosphate (100 mM) | NEB |
| Uridine-5′-triphosphate (100 mM) | NEB |
| DL-dithiothreitol (DTT) (1M) | Sigma-Aldrich |
| Spermidine (100 mM) | Sigma-Aldrich |
| Pyrophosphatase, inorganic (100 U/mL) | NEB |
| RNase inhibitor, human (40,000 U/mL) | NEB |
| T7 RNA polymerase (50,000 U/mL) | NEB |
| Linear DNA template (1 mg/mL) | — |

Results: The twelve T7 RNA polymerase variants, their corresponding sequences, and the concentrations of RNA synthesized by the variants are listed in Table 4. The RNA yield varied from 0.13 mg/mL (variant 3) to 6.66 mg/mL (variant 5 and 9). The total RNA yield from the wild-type T7 RNA Polymerase was 3.13 mg/mL. Compared to the wild-type T7, there is an increase in total RNA yield in variants 1, 2, 4, 5, 6, 8, 9, and 12. Variants 5 and 9 yielded the highest concentration at 6.66 mg/mL.

TABLE 4
Total RNA yield from T7 RNA Polymerase variants

| | Mutation | Mutation | Total | Fold |
|---|---|---|---|---|
| T7 RNA | (relative to | (relative to | RNA | Increase |
| Polymerase | SEQ ID NO: | SEQ ID NO: | yield | Over |
| variants | 1) | 2) | (mg/mL) | Wild Type |
| 1 | Q64H | Q57H | 6.52 | 2.08 |
| 2 | E69P | E62P | 6.53 | 2.08 |
| 3 | D427C | D420C | 0.13 | — |
| 4 | M437F | M430F | 3.91 | 1.25 |
| 5 | E649F | E642F | 6.66 | 2.12 |
| 6 | W688K | W681K | 6.17 | 1.97 |
| 7 | A257M | A250M | 0.96 | — |
| 8 | K779A | K772A | 6.41 | 2.05 |
| 9 | I20A | I13A | 6.66 | 2.12 |
| 10 | I784M | I777M | 0.54 | — |
| 11 | D659T | D652T | 0.25 | — |
| 12 | G681P | G674P | 6.14 | 1.96 |
| Wild-type T7 | — | — | 3.13 | — |
| RNA Polymerase | | | | |

### Example 6: RNA Integrity of Twelve T7 RNA Polymerase Variants

The twelve T7 RNA polymerase variants, their corresponding mutation, and the integrity of synthesized RNA are listed in Table 5. RNA samples from IVT reactions were analyzed on a Beckman Coulter PA 800plus equipped with a LIF detector using a bare fused-silica capillary pre-installed cartridge. RNA integrity (%) was assessed by normalizing the integrated intensity of the main sample peak to the integrated intensity of all peaks in the electropherogram.

TABLE 5
RNA integrity as determined by Capillary gel electrophoresis

| T7 RNA | Mutation | Mutation | RNA |
|---|---|---|---|
| Polymerase | (relative to | (relative to | Integrity |
| variants | SEQ ID NO: 1) | SEQ ID NO: 2) | (%) |
| 1 | Q64H | Q57H | 53 |
| 2 | E69P | E62P | 47 |
| 3 | D427C | D420C | n.d. |
| 4 | M437F | M430F | 63 |
| 5 | E649F | E642F | 58 |
| 6 | W688K | W681K | 62 |
| 7 | A257M | A250M | 56 |
| 8 | K779A | K772A | 62 |
| 9 | I20A | I13A | 51 |
| 10 | I784M | I777M | 54 |
| 11 | D659T | D652T | n.d. |
| 12 | G681P | G674P | 62 |
| Wilt-type T7 RNA | — | — | 58 |
| polymerase | | | |

Results: The integrity of RNA synthesized from the wild-type T7 RNA polymerase was 58%, while the RNA integrity from T7 RNA polymerase variants varied from 4700 to 63%. RNA from variants 1, 2, 7, 9, and 10 had lower integrity than the wild type, while variants 4, 6, 8, and 12 generated RNA with higher integrity. The integrity of RNA from variants 3 and 11 could not be determined because of the low yield of RNA from these variants. Taking into consideration the yield and the integrity of RNA, K779A was selected as one of the backbone candidates for the second round of library preparation.

### Example 7: Testing the Efficiency of Another Set of T7 RNA Polymerase Variants

Additional vectors encoding T7 RNA polymerase variant were prepared using methods known to a skilled person (e.g., Example 1). T7 RNA Polymerase variant cultures were plated onto LB agar plates with 1% glucose and 34 ug/ml chloramphenicol and were grown overnight at 37° C. A single colony was inoculated into 50 ml of LB broth with 1% glucose and 30 ug/ml chloramphenicol. The cultures were grown for 18-20 hours at 30° C., 250 rpm, and sub-cultured at a dilution of approximately 1:20 into 1000 ml of Terrific Broth with 30 ug/ml of chloramphenicol. The cultures were incubated for approximately 3 hours at 30° C., 250 rpm, to an OD600 of 0.6-0.8, and then induced with IPTG at a final concentration of 1 mM. The induced cultures were incubated for an additional 20 h at 30° C., 250 rpm. Following this incubation period, the cultures were centrifuged at 4000 rpm for 10 min. The culture supernatant was discarded, and the cell pellets were frozen. Frozen cell pellets (approx. 1 g each) were resuspended in 40 mLs Lysis buffer (50 mM HEPES, pH 7.5, 300 mM NaCl, 0.1 mM TCEP, Roche EDTA-free protease inhibitors). The cell suspension was chilled in an ice bath and lysed using a sonicator (QSonica). The crude lysate was pelleted by centrifugation (30,000 RCF for 30 min at 4° C.), and the supernatant was then gently mixed with 1 mL of complete His-tag purification resin at 4° C. for 2 hours. The resin was then collected in a gravity flow column, washed (Wash Buffer: 20 mM HEPES, pH 7.5, 300 mM NaCl, 0.1 mM TCEP, 45 mM imidazole) and protein was eluted (Elution Buffer: 20 mM HEPES, pH 7.5, 300 mM NaCl, 0.1 mM TCEP, 300 mM imidazole). The eluates were then purified using size-exclusion chromatography (Column: HiLoad 16/600 Superdex 200 pg; Cytiva) on an AKTA Pure FPLC system utilizing an Alias autosampler. Samples were sequentially run over the column and peaks were collected. The sizing buffer was 2×T7RNAP storage buffer (100 mM Tris HCl pH 8.0, 200 mM NaCl, 2 mM DTT). For all 10 T7RNAP samples, peaks collected were combined with an equal volume of 100% glycerol (50% final concertation). Enzyme concentrations in the preparations were measured by absorption at 280 nm, and analysis by SDS-PAGE and LC-MS/MS was performed (data not shown).

Eight T7 RNA polymerase variants were tested in IVT reactions along with the wild-type T7 RNA polymerase. All IVT reactions were carried out according to standard methods using components listed in Table 3. Reactions were initiated by the addition of a linearized DNA template (˜6400 nucleotides in length) followed by incubation at 37° C. for 4 hours. DNase was added and incubated further for 1 hour. Reactions were then quenched by the addition of Lithium chloride (LiCl) and storing the reactions at −20° C. to precipitate synthesized RNA. Following LiCl precipitation, RNAs were rehydrated in nuclease-free water. In this experiment, T7 RNA polymerase variants were compared with the Wild type T7 RNA polymerase. Samples yields were measured by Qubit™ BR assay kit according to the provided protocol. The integrity of the samples was measured by LabChip GX II touch characterization system (PerkinElmer) according to the provided protocol (Table 6).

TABLE 6
Total RNA yield and % RNA Integrity of Eight T7 RNA Polymerase variants

| | | | Total | Fold | |
|---|---|---|---|---|---|
| T7 RNA | Mutation | Mutation | RNA | Increase | RNA |
| Polymerase | (Relative to | (Relative to | yield | Over Wild | Integrity |
| variants | SEQ ID NO: 1) | SEQ ID NO: 2) | (mg/mL) | Type | (%) |
| 13 | M437F | M430F | 2.12 | — | 92.42 |
| 14 | K779A | K772A | 3.93 | 1.40 | 94.07 |
| 15 | I20A | I13A | 3.38 | 1.20 | 91.89 |
| 16 | G681P | G674P | 3.76 | 1.34 | 89.93 |
| 17 | K779A, N15A, | K772A, N8A, I13N, | 4.33 | 1.54 | 88.69 |
| | I20N, Q655N, and | Q648N, and D652E | | | |
| | D659E | | | | |
| 18 | K779A, W688Q, | K772A, W681Q, | 3.50 | 1.25 | 95.75 |
| | and F761K | and F754K | | | |
| 19 | K779A, A264F, | K772A, A257F, | 5.34 | 1.90 | 88.89 |
| | A268C, and N425G | A261C, and N418G | | | |
| 20 | K779A, A264V, | K772A, A257V, | 4.98 | 1.77 | 91.31 |
| | T381L, and N425G | T374L, and N418G | | | |
| Wild-type | — | — | 2.80 | — | 92.75 |
| T7 RNA | | | | | |
| Polymerase | | | | | |

Results: Eight T7 RNA polymerase variants, their corresponding sequences, the concentration of T7 RNA polymerase variants (total RNA yield), and the % RNA integrity of T7 RNA polymerase variants are listed in Table 6. Variants 13-20 were assessed for their efficiency in in vitro transcription of mRNA samples (4 kb in size). The RNA yield varied from 2.12 mg/mL (variant 13) to 5.34 mg/mL (variant 19). The total RNA yield from the wild-type T7 RNA Polymerase was 2.80 mg/mL. Compared to the wild-type T7 polymerase, there is an increase in total RNA yield in variants 13-20. Also, variants 14 and 18 demonstrate a higher % RNA integrity than the wild-type T7 polymerase.

### Example 8: dsRNA Content in RNAs Transcribed Using Variants of T7 RNA Polymerase

% Double stranded RNA (% dsRNA) in each of the RNA samples generated from IVT reactions using the disclosed variants was measured by Luminex-based sandwich capture assay according to the provided protocol (e.g., xMAP® capture sandwich immunoassay, Luminex®). Known volumes of antibody-coupled beads were added to the wells of a bead plate. IVT generated RNA samples were added to the beads, and the samples were incubated for 30 minutes at room temperature on a plate shaker. After the incubation, plates were washed with assay buffer (PBS-TBN). A labeled detection antibody was added to the wells, followed by 30 minutes of incubation and washes. At the end of the reaction, signals from each of the wells were read on a Luminex FlexMAP 3D instrument. Total RNA was measured using the Qubit™ BR assay kit, and dsRNA was measured using the Luminex sandwich assay. Using these two measurements pg dsRNA/ug RNA or % dsRNA was calculated (Table 7).

TABLE 7
% Double Stranded RNA in T7 RNA Polymerase variants

| T7 RNA | Mutation | Mutation | |
|---|---|---|---|
| Polymerase | (Relative to | (Relative to | % |
| variants | SEQ ID NO: 1) | SEQ ID NO: 2) | dsRNA |
| 13 | M437F | M430F | 0.023 |
| 14 | K779A | K772A | 0.012 |
| 15 | I20A | I13A | 0.017 |
| 16 | G681P | G674P | 0.017 |
| 17 | K779A, N15A, , | K772A, N8A, | 0.023 |
| | I20N, Q655N | I13N, Q648N, | |
| | and D659E | and D652E | |
| 18 | K779A, W688Q, | K772A, W681Q, | 0.020 |
| | and F761K | and F754K | |
| 19 | K779A, A264F, | K772A, A257F, | 0.023 |
| | A268C, and N425G | A261C, and N418G | |
| 20 | K779A, A264V, | K772A, A257V, | 0.020 |
| | T381L, and N425G | T374L, and N418G | |
| Wild-type T7 | — | — | 0.020 |
| RNA | | | |
| Polymerase | | | |

Results: The dsRNA (% dsRNA) content in RNAs transcribed using the eight variants of T7 RNA Polymerase are shown in Table 7. Compared to wild-type T7 RNA polymerase, variants 14-16 had lower dsRNA content. % dsRNA in variants 18 and 20 is the same as the wild-type T7 RNA polymerase.

A person of ordinary skill in the art would recognize that provided T7 RNA polymerase variants are superior to the wild-type T7 RNA polymerase at least in the following aspects: yield, % integrity, % ds DNA contaminants, among other things.

1. A T7 ribonucleic acid (RNA) polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:1 or a functional fragment thereof, wherein the T7 RNA polymerase amino acid sequence includes the feature that: at least one amino acid selected from the group consisting of amino acid N15, I20, Q64, E69, A257, A264, A268, T381, N425, D427, M437, E649, Q655, D659, G681, W688, F761, K779, and I784 is substituted to a different amino acid to that found at the corresponding amino acid position in SEQ ID NO:1.

2. The T7 RNA polymerase or functional fragment thereof of claim 1, wherein the amino acid sequence comprises at least one feature selected from the group consisting of: N15 is substituted with A or T or Y or Q or S, I20 is substituted with A or L or V or G or M or N, Q64 is substituted with N or S or T or H, E69 is substituted with D or P, A257 is substituted with M or L or V or I or G, A264 is substituted with F or V or M or L or I or G, A268 is substituted with C or M or L or I or G, T381 is substituted with L or Y or N or Q or S, N425 is substituted with G or T or Y or Q or S, D427 is substituted with E or C, M437 is substituted with A or L or V or I or G or F or D, E649 is substituted with D or F, Q655 is substituted with N or T or Y or S, D659 is substituted with E or T, G681 is substituted with A or L or V or I or M or P, W688 is substituted with H or Y or F or K or Q, F761 is substituted with K or W or Y or H, K779 is substituted with R or A, and I784 is substituted with A or L or V or M or G.

3. The T7 RNA polymerase or functional fragment thereof of claim 1, wherein the amino acid sequence comprises at least one feature selected from the group consisting of: N15 is substituted with A, I20 is substituted with A, I20 is substituted with N, Q64 is substituted with H, E69 is substituted with P, A257 is substituted with M, A264 is substituted with F, A264 is substituted with V, A268 is substituted with C, T381 is substituted with L, N425 is substituted with G, D427 is substituted with C, M437 is substituted with F, M437 is substituted with D, E649 is substituted with F, Q655 is substituted with N, D659 is substituted with T, D659 is substituted with E, G681 is substituted with P, W688 is substituted with K, W688 is substituted with Q, F761 is substituted with K, K779 is substituted with A, and I784 is substituted with M.

4. The T7 RNA polymerase or functional fragment thereof of claim 1, wherein the amino acid sequence comprises at least one feature selected from the group consisting of: Q64 is substituted with H, E69 is substituted with P, N15 is substituted with A, I20 is substituted with N, I20 is substituted with A, A264 is substituted with F, A264 is substituted with V, A268 is substituted with C, T381 is substituted with L, M437 is substituted with F, M437 is substituted with D, N425 is substituted with G, E649 is substituted with F, Q655 is substituted with N, D659 is substituted with E, G681 is substituted with P, W688 is substituted with Q, F761 is substituted with K, and K779 is substituted with A.

5. (canceled)

6. The T7 RNA polymerase or functional fragment thereof of claim 4, wherein the amino acid sequence comprises at least one feature selected from the group consisting of: M437 is substituted with F, I20 is substituted with A, G681 is substituted with P, and K779 is substituted with A.

7. (canceled)

8. The T7 RNA polymerase or functional fragment thereof of claim 1, wherein at least two, at least three, at least four, at least five, at least six, at least ten, or at least twenty amino acids are substituted to a different amino acid to that found at the corresponding amino acid position in SEQ ID NO:1.

9. The T7 RNA polymerase or functional fragment thereof of claim 8, wherein the amino acid substitution comprises: (a) K779A and at least one other amino acid substitution; (b) M437F and at least one other amino acid substitution; (c) I20A and at least one other amino acid substitution; (d) G681P and at least one other amino acid substitution; (e) N15A and at least one other amino acid substitution; (f) I20N and at least one other amino acid substitution; (g) Q655N and at least one other amino acid substitution; (h) D659E and at least one other amino acid substitution; (i) W688Q and at least one other amino acid substitution; (j) F761K and at least one other amino acid substitution; (k) A264F and at least one other amino acid substitution; (l) A264V and at least one other amino acid substitution; (m) A268C and at least one other amino acid substitution; (n) N425G and at least one other amino acid substitution; and (o) T381L and at least one other amino acid substitution.

10. (canceled)

11. The T7 RNA polymerase or functional fragment thereof of claim 8, wherein the amino acid substitution comprises K779A, N15A, I20N, Q655N, and D659E.

12. The T7 RNA polymerase or functional fragment thereof of claim 8, wherein the amino acid substitution comprises K779A, W688Q, and F761K.

13. The T7 RNA polymerase or functional fragment thereof of claim 8, wherein the amino acid substitution comprises: (a) K779A, A264F, A268C, N425G; (b) K779A, A264V, T381L, N425G; or (c) a combination thereof.

14. (canceled)

15. The T7 RNA polymerase of claim 1, wherein the T7 RNA polymerase is full length.

16. A T7 RNA polymerase comprising an amino acid sequence that is at least 80%, 85%, 90%, 91%, 92%, 93%, 94%, 95%, 96%, 97%, 98%, or 99% identical to the amino acid sequence set out in SEQ ID NO:2 or a functional fragment thereof, wherein the T7 RNA polymerase amino acid sequence includes the feature that: at least one amino acid selected from the group consisting of amino acid N8, I13, Q57, E62, A250, A257, A261, T374, N418, D420, M430, E642, Q648, D652, G674, W681, F754, K772, and I777 is substituted to a different amino acid to that found at the corresponding position in SEQ ID NO: 2.

17. The T7 RNA polymerase or functional fragment thereof of claim 16, wherein the amino acid sequence comprises at least one feature selected from the group consisting of: N8 is substituted with A or T or Y or Q or S, I13 is substituted with A or L or V or G or M or N, Q57 is substituted with N or S or T or H, E62 is substituted with D or P, A250 is substituted with M or L or V or I or G, A257 is substituted with F or V or M or L or I or G, A261 is substituted with C or M or L or I or G, T374 is substituted with L or Y or N or Q or S, N418 is substituted with G or T or Y or Q or S, D420 is substituted with E or C, M430 is substituted with A or L or V or I or G or F or D, E642 is substituted with D or F, Q648 is substituted with N or T or Y or S, D652 is substituted with E or T, G674 is substituted with A or L or V or I or M or P, W681 is substituted with H or Y or F or K or Q, F754 is substituted with K or W or Y or H, K772 is substituted with R or A, and I777 is substituted with A or L or V or M or G.

18. (canceled)

19. A polynucleotide encoding the T7 RNA polymerase or functional fragment thereof, as claimed in claim 1.

20. A vector comprising the polynucleotide of claim 19.

21. A host cell comprising the vector of claim 20.

22. A composition comprising the T7 RNA polymerase or functional fragment thereof of claim 1.

23. (canceled)

24. (canceled)

25. A method of performing an in vitro transcription (IVT) reaction, comprising combining a DNA and nucleoside triphosphates with the T7 RNA polymerase of claim 1.

26. The method of claim 25, wherein the nucleoside triphosphates comprise modified nucleoside triphosphates.

27. (canceled)

28. (canceled)

29. (canceled)

30. (canceled)
